(19) United States
(12) Patent Application Publication
(10) Pub. No.: US 2020/0158804 A1
(43) Pub. Date: May 21, 2020

(54) MR SEQUENCE GENERATION

(71) Applicant: Siemens Healthcare GmbH, Erlangen (DE)

(72) Inventor: Thomas Kluge, Hirschaid (DE)

(21) Appl. No.: 16/684,557

(22) Filed: Nov. 14, 2019

(30) Foreign Application Priority Data
Nov. 15, 2018 (EP) 18206365.1

(51) Int. Cl. G01R 33/54; A61B 5/055

(52) U.S. Cl. CPC G01R 33/546; A61B 5/055

(57) ABSTRACT

A method for generating control commands for execution on a magnetic resonance (MR) scanner includes receiving an MR sequence via an input interface, partitioning the MR sequence into a sequence of time slices, and defining an extendable set of sequence block control data types. Each time slice is mapped to at least one instance of a sequence block control data type or to at least one instance of a fundamental control data type for providing a stream of events. An event handler is provided for each event. A dispatcher is accessed for reading each event in the provided stream of events and passing each event together with the dispatcher to an appropriate event handler, so that the dispatcher may be called recursively by the event handlers. Fundamental control data types are translated into concrete control commands of the particular scanner to be used for execution on the MR scanner.

This application claims the benefit of European Patent Application No. EP18206365.1, filed on Nov. 15, 2018, which is hereby incorporated by reference in its entirety.

### BACKGROUND

The present embodiments relate to generating control commands for magnetic resonance (MR) scanner pulse sequences for acquiring imaging information using a magnetic resonance scanner.

Measurement sequences are necessary to control an MR scanner.

For example, the generation of MR images in an MR scanner requires an exact, temporal correlation between the radio-frequency excitation of the spins, the spatial coding, and the detection of the resonant response of the spins. The temporal correlation of the excitation, preparation, and detection is a pulse sequence or a measurement sequence. A single segment of the measurement sequence may be classified according to a corresponding purpose. This division is designated as a time slice. A time slice is thus an established time segment within the measurement sequence that serves a specific purpose (e.g., the excitation of the nuclear spins). Time slices contain control instructions for the MR scanner. The time slices precisely determine what the MR scanner has to do and at which point in time. A series of time slices may be executed in the MR scanner because the MR scanner contains all instructions and a corresponding temporal correlation to the control of the MR scanner. Alternatively, the execution of the time slices with a control unit connected with the MR scanner is also possible.

A measurement sequence may be executed as a series of time slices in the MR scanner. Within the measurement sequence for execution in an MR scanner, the time slices are joined seamlessly (e.g., without gaps). Each of the time slices has a specific length, and at least one pulse with a pulse shape is associated with every time slice. Each of the time slices may be associated with a type from the set: transmission type for transmission of RF power; reception type to detect the resonant response of the nuclear spins; and warp type to prepare the nuclear spins. Time slices of the transmission type are used as excitation pulses to excite the nuclear spins, to refocus the nuclear spins, and in a hybrid form that serves both for excitation and for refocusing. In addition to these, there are time slices in which RF energy is emitted and/or an RF signal is received. RF pulses are known for the inversion of the nuclear magnetization, called inversion pulses.

The generation of different MR sequences or measurement sequences is necessary in order to generate MR images with the appropriate diagnostic information. For example, it is possible to significantly affect the contrast of an image by the selection of a proper measurement sequence. The preparation of the spin system (e.g., using pulses for RF excitation, gradient pulses, wait times) has a decisive influence on the quality and property of the acquired MR image.

Usually, measurement sequences for execution in an MR scanner may be prepared by a sequence programmer (e.g., in one of the known programming languages, like C++), describing all properties and the temporal sequence of the time slices in a completely parameterized form and with permissible parameter values for execution in the MR scanner.

A measurement sequence for execution in an MR scanner in a clinical everyday situation typically is composed of 1000 to 1000000 time slices that are involved in a sensitive temporal correlation among one another.

Thus, a pulse sequence in the context of magnetic resonance scanners may be defined as the sequence of control instructions for the acquisition of imaging information in the magnetic resonance (MR) scanner. The pulse sequence may, for example, include one or more radio frequency (RF) pulses to be radiated into the tissue of a subject, together with instructions controlling a magnetic field surrounding the subject, such as a direction, strength, and structure (e.g., gradient) thereof. Parameters of the pulse sequence are known in the art and include, for example, duration or measurement time, total energy, power, and a parameter defined within the scope of the temporal subrange, such as an amplitude, a duration, and a shape of gradient and RF pulses, or coil settings for acquisition procedures. An overview of computer-aided MR imaging (MRI) pulse sequence generation is given in, for example, “Handbook of MRI Pulse Sequences” by M. Bernstein et al, Elsevier, 2004, with basic pulse functions in Chapter 3, p. 67 to 84, for example.

It is known to create pulse sequences from multiple sequence building blocks, which are high in number and complexity, such that a manual combination and contextual redevelopment of methods requires a large amount of resources in development, methodological expertise, and quality assurance. Generating MR sequences by combining sequence blocks is challenging due to the dependency depth of parameters associated with the respective sequence blocks since methods and parameter dependencies (e.g., dependencies necessary for calculating the respective sequence block) are hard to abstract. More precisely, the operator is to know exactly which parameters the respective sequence block depends on to have that respective parameter readily available at the time of calculating the sequence block.

Generally, there are a plurality of different scanner types, including scanners with different functionalities, with different hardware and/or software, with different technical resources (e.g., coils, magnetic field strength, gradient strength, and switching speed, number of receive/transmit coils, hardware duty cycles, reconstruction speed, and memory capacity). All these kind of different scanner types are to be considered when generating MR sequences.

For developing MRI sequences, it is known in state of the art to use computer programs that assist in defining MRI sequences.

### SUMMARY AND DESCRIPTION

The scope of the present invention is defined solely by the appended claims and is not affected to any degree by the statements within this summary.

The existing programs are complex and cumbersome. Known programming paradigms are based on a monolithic concept and are not or only partly adapted to the specific use case. There is a need to provide a program that is simpler to be used and may be extended easily. Further, existing programs are architecture specific and are to be recompiled whenever the sequence is ported to a new scanner type or just to a new software version of the same scanner. Unfortunately, there is no abstraction level separating the control of the scanner hardware from the physical definition of the events of the pulse sequence.

The present embodiments may obviate one or more of the drawbacks or limitations in the related art. For example, a solution for making magnetic resonance imaging (MRI) sequence generation easier, more efficient, and more flexible, so that generated sequences may be ported easily to other scanner types is provided. Further, performance may be increased for a resource intensive complex task.

In one embodiment, a method for generating control commands for execution on a plurality of different magnetic resonance (MR) scanner types, by a system, by a computer program according to one or more of the present embodiments is provided. Advantageous aspects, features, and embodiments are described below.

In the following, the present embodiments are described with respect to a system as well as with respect to a method. Features, advantages, or alternative embodiments herein may be assigned to the other objects (e.g., the computer program or a computer program product), and vice versa. In other words, the subject matter that is described with respect to the system may be improved with features described in the context of the method, and vice versa. In this case, the functional features of the method are embodied by structural units of the system and vice versa, respectively.

One aspect relates to a method for generating control commands for execution on an MR scanner. The MR scanner may be of any different type. The method includes receiving a provided stream of events representing the received MR sequence to be executed. An event handler is provided for each event type. Each of the event handlers is adapted for processing at least one event type. A dispatcher is accessed for reading each event in the provided stream of events, and each event is passed together with the dispatcher itself to an appropriate event handler, so that the dispatcher may be called recursively by the event handlers for generating control commands to be used for execution on the MR scanner.

The present embodiments provide technical advantages. The suggested solution provides an extendable data-driven interface. Thus, it is not necessary to provide complex software interfaces, which is typically more complex and more difficult to understand.

With this system, method, and program, a maximal flexibility may be achieved with respect to a level of abstraction of the control data.

The system and method and program provide a high degree of modularity and decouple control of scanner hardware from the physical definition of the events, representing the MR sequence.

Further, the system, method, and program provide a maximum degree of scalability due to the option to define new events and event handlers.

According to an embodiment, the method may further include the following acts that are directed for the provisioning or the stream of events. An MR sequence is received via an input interface, and the MR sequence is partitioned in a sequence of time slices. Each time slice of the sequence of time slices is a continuous uninterrupted period of time within the MR sequence. An extendable set of sequence block control data types is defined. A sequence block control data type represents a formalized (e.g., generic and standardized) and a self-contained description of a time slice of the MR sequence. Each time slice is mapped to an instance of a sequence block control data type or an instance of a fundamental control data type for providing the stream of events.

According to another embodiment, the event in the stream of events is a package consisting of a uniform unique identifier (UUID), a data size in bytes, and the event data itself. The UUID and the data size are accessible separately from the event data. A UUID may be for identifying datasets or information in computer systems. The UUID may be a 128-bit number. UUIDs are documented as part of ISO/IEC 11578:1996 “Information technology—Open Systems Interconnection—Remote Procedure Call (RPC),” and more recently, in ITU-T Rec. X.667|ISO/IEC 9834-8:2005. With this feature, each of the events is addressable in a dedicated manner. One first event may be unambiguously differentiated from any other second event that potentially could be sent to the MR sequence. In one embodiment, the assignment between event and an appropriate event handler is realized by including the respective QUID in the event and within the dispatcher assigning this QUID to an event handler. Fundamental control data types and the sequence block control data types may each be identified in a uniform and unique manner and, for example, by such a UUID.

According to another embodiment, the recursive calls to the dispatcher may be used for applying a composite design pattern or a tree structure for generating single time slices or the complete MR sequence. This improves efficiency of the solution with respect to re-use of existing time slice types (e.g., respiratory event) and event stream data rate.

It is an advantage that the generated control commands are scanner-independent. Thus, the generated control commands may be used for execution on a variety of different scanners and platforms. The generated control commands may be translated into scanner-specific control commands by the respective appropriate event handler for execution on a particular scanner.

According to another embodiment, the dispatcher and the event handler are both implementing the same interface for sequence block execution. With this feature, recursive calls of the dispatcher are possible, which leads to a high degree of flexibility and efficiency.

According to another embodiment, the definition of the set of sequence block control data types or the providing of the events is based on a provided abstract model for MR sequences. The abstract model may be stored in a data storage that is accessible via a network connection. For example, a model that is described in U.S. Patent Application Publication No. 2010/0090694 (DE 10 2008 044 828 B3), the contents of which are entirely incorporated by reference herein, may be used. The model may be based on an extendable set of fundamental control data types.

To provide a maximum degree of flexibility, the set of fundamental control data types and/or the set of sequence block control data types may be extended. Thus, the set of sequence block control data types may be extended easily by adding new control data types, and the set of fundamental control data types may be extended by adding new fundamental control data types. Both are possible without affecting existing code.

The highest level of scalability is provided by making the set of event handlers extendable. For example, the set of event handlers may be extended or adapted easily by adding new event handlers without affecting existing code.

In an embodiment, the stream of events may be provided in a standardized format. The standardized format may be selected in a configuration phase. Possible options for such a standardized format are Json, hierarchical data format (HDF), or XML, or, most efficiently, is a byte stream (e.g., that may be generated from any of the mentioned formats) as a stream of 128 bit UUID−64 bit data size (=NB)−NB bytes.

In another embodiment, the acts of mapping, which corresponds to a serialization of the MR sequence, accessing the dispatcher, and calling the event handlers recursively are implemented by an IRecursiveEventHandler interface, which is a pure virtual or abstract base class in C++.

With this standardized format in mind, the interface for the recursive event handler, the IRecursiveEventHandler, in C++ may look like:

class IRecursiveEventHandler
{
public:

virtual void executeSequenceBlock(

IRecursiveEventHandler &rRecursiveEventHandler,
const UUID_t &rUUID,
ConstMemPtr_t pMem,
MemSize_t MemSize,
ConstMemPtr_t pData,
MemSize_t DataSize
) = 0;

virtual ~IRecursiveEventHandler ( ) = default;

};

In another embodiment, the stream of events may be provided by a software Plug-In into the MR scanner control software. The Plug-In is written for this specific purpose. The Plug-In interface is configured for this purpose. The event handlers are also plug-ins via IRecursiveEventHandler interface. This together with the generic event stream data format leads to the high scalability.

Another aspect relates to a computing unit for generating control commands for execution on an MR scanner. The computing unit includes an input interface for receiving a provided stream of events, and an event handler that is provided for each event. The computing unit also includes a dispatcher for reading each event in the provided stream of events and passing each event together with the dispatcher itself to an appropriate event handler, so that the dispatcher may be called recursively by the event handlers for generating control commands to be used for execution on the MR scanner. Each of the event handlers is configured for processing one or more specific events. The input interface, the event handler, and/or the dispatcher may be formed by one or more processors.

The computing unit may include or interact with a second computing unit. The second computing unit may include modules that are responsible for providing the stream of events. The modules may include an input interface for receiving an MR sequence, and a partitioning unit that is configured for partitioning the received MR sequence in a sequence of time slices. Each time slice is a continuous uninterrupted period of time within the MR sequence. The modules may also include first storage (e.g., a first memory) for providing an extendable set of sequence block control data types. A sequence block control data type represents a formalized and self-contained description of a time slice of an MR sequence. The modules also include a mapping unit that is adapted for mapping each time slice to an instance of a sequence block control data type or to an instance of a fundamental control data type for providing a stream of events representing the received MR sequence. In one embodiment, one or more of the modules are formed by one or more processors.

Another aspect relates to a system for generating control commands for execution on an MR scanner. The system includes at least one MR scanner to be operated with control commands to be generated. The scanner may be of any type (e.g., without any requirements with respect to hardware and software, and even scanners from different manufacturers may be addressed). The system includes an input interface for receiving an MR sequence, and a partitioning unit that is configured for partitioning the received MR sequence in a sequence of time slices. Each time slice is a continuous uninterrupted period of time within the MR sequence. The system also includes a first storage (e.g., a first memory) for providing an extendable set of sequence block control data types. A sequence block control data type represents a formalized and self-contained description of a time slice of an MR sequence. The system includes a mapping unit that is configured for mapping each time slice to an instance of a sequence block control data type or to an instance of a fundamental control data type for providing a stream of events representing the received MR sequence. An event handler is provided for each event. Each of the event handlers is adapted for processing one specific event type. A dispatcher is provided for reading each event in the provided stream of events and passing each event together with the dispatcher itself to an appropriate event handler, so that the dispatcher may be called recursively by the event handlers for generating control commands to be used for execution on the MR scanner. The input interface, the partitioning unit, the mapping unit, the event handler, and/or the dispatcher may be formed by one or more processors.

Another aspect relates to a computer program for generating an MR sequence or parts thereof. The computer program includes program code for causing a computing entity to carry out the method as described above, when the computer program is run on the computing entity.

In the following, a definition of terms, used within this document is given.

A sequence block control data type describes an electronic digital dataset, representing a formalized, abstract, generic, and a self-contained description of a time slice of the MR sequence. The sequence block control data types are independent of the hardware, software, and physical scanner properties. In a possible embodiment, the types may be proprietary. Possible examples of a type are: slice selective excitation; readout with constant gradient; warp block; FLASH Kernel; TSE Kernel; inversion recovery; regional saturation; and spectral selective saturation.

A fundamental control data type describes an electronic digital dataset. Fundamental control data types in contrast to sequence block control data types are on a sub-time-slice-level (e.g., are used for building a complete time slice). Fundamental control data types represent physical capabilities of the MR scanner (e.g., on an abstract level). A fundamental control data type may be construed as a low level command. Examples of a fundamental control data type are: Initialize a time slice; finalize a time slice; define duration of the time slice; insert an RF pulse (Bl-field) into the time slice; insert a gradient pulse (Bz-field) into the time slice; and insert a read-out interval into the time slice.

An event is an electronic dataset, representing a part of the MR sequence in a modeled manner. An event is either an instance of a sequence block control data type or an instance of a fundamental control data type.

An event handler is implemented as software for processing a particular event. An event handler may be implemented as a class implementing the IRecursiveEventHandler. One type of event handlers may be configured to process sequence block control data types, and another type of event handlers may be configured to process fundamental control data types. Only the event handlers for fundamental control data types are communicating directly with the scanner interface or output interface. In an embodiment, only the fundamental control data types are to be translated into scanner specific control commands.

A dispatcher may be implemented in software (e.g., C++). The functionality of the dispatcher is to control the processing workflow. The dispatcher processes event after event within the stream of events. For processing a particular event, the dispatcher passes the event together with the dispatcher itself to an associated event handler.

The control commands may be used to generate a MR sequence on a particular MR scanner. A control command may be construed as an event.

The input interface for receiving an MR sequence may be an application programming interface (API).

The input for the dispatcher (e.g., the stream of events) is provided by an exchangeable software plug-in with a specific interface.

Another aspect relates to a computer program product including a computer program. The computer program is loadable into a memory unit of a computing unit. The computer program includes program code sections to make the computing unit execute the method as described above, when the computer program is executed in the computing unit.

Another aspect relates to a computer-readable medium, on which program code sections of a computer program are stored or saved. The program code sections re loadable into and/or executable in a computing unit to make the computing unit execute the method as described above, when the program code sections are executed in the computing unit.

The realization of one or more of the present embodiments by a computer program product and/or a computer-readable medium has the advantage that already existing computing units (e.g., servers, clients, or the MR scanners itself) may be easily adopted by software updates to work as proposed by the present embodiments.

The properties, features, and advantages of the present embodiments described above, as well as the manner the properties, features, and advantages are achieved, become clearer and more understandable in the light of the following description and embodiments, which will be described in more detail in the context of the drawings. This following description does not limit the invention to the described embodiments. Same components or parts may be labeled with the same reference signs in different figures.

An embodiment may also be any combination of the embodiments.

These and other aspects of the invention will be apparent from and elucidated with reference to the embodiments described hereinafter.

### BRIEF DESCRIPTION OF THE DRAWINGS

FIG. 1 schematically and exemplarily shows a magnetic resonance (MR) scanner that is operated by an associated computing entity; and

FIG. 2 is a flow chart of one embodiment of a method for generating control commands to be used for different MR scanner types.

### DETAILED DESCRIPTION

The method according to the present embodiments implements a sequence run functionality. For example, the method provides a translation or transformation of hardware- and scanner-independent event streams into hardware- and scanner specific control commands to be executable on a magnetic resonance (MR) scanner. The method may also be used for providing a (back) translation of scanner specific control commands into an abstracted form, into a scanner-independent event stream. The scanner specific control commands may be construed as measurement order.

An event is modeled in a standardized generic manner. The event is marked with a unique identifier (e.g., an UUID, see https://de.wikipedia.org/wiki/Universally_Unique_Identifier) to unambiguously differentiate the event from any other event that potentially may be sent to the sequence. The event then provides a complete self-contained description of a continuous uninterrupted period of time. So, the sequence (e.g., a continuous series of continuous uninterrupted periods of time) in the time domain may be directly mapped to a stream of events. A compact format for the events (e.g., when it comes to serialization of the event stream, the execution of the event stream in a real-time program, the expandability with new event types, and the invariance in different programming languages) is a simple standardized binary format that, for example, consists of a UUID, a data size in bytes, and the event data itself. Within the data section, any content may be freely transported.

The first implementation of IRecursiveEventHandler is the sequence execution function itself (e.g., the sequence execution function is an IRecursiveEventHandler), which is the root event handler dispatching events to other implementations. The first implementation of IRecursiveEventHandler may be programmed in a way that allows to simply add an arbitrary number of other implementations of IRecursiveEventHandler to the sequence execution and map each of those implementations to one or more event UUIDs. The mapping of a UUID to a handler may be unique to be able to unambiguously route a particular event to a distinct handler.

By passing the event handler itself recursively to the event handling function, each implementation of IRecursiveEventHandler may make use of the root event dispatcher.

Any known functionality may be implemented as an IRecursiveEventHandler. For example, the functionality execute a complete existing sequence may be implemented as an IRecursiveEventHandler. The event type would be the existing MR protocol structure. As another example, the functionality execute events read from a file may be implemented as an IRecursiveEventHandler. The event type would be the file path. As yet another example, the functionality execute events read from a remote computer via network may be implemented as an IRecursiveEventHandler. The event type would be the connection details for the remote computer. As an example, the functionality execute user defined loops structure and produce events that control other IRecursiveEventHandler implementations may be implemented as an IRecursiveEventHandler. This way, the existing source code may be structured much more than the existing source code is today. As another example, the functionality a time slice sending an RF pulse over a constant gradient may be implemented as an IRecursiveEventHandler. As yet another example, the functionality a time slice sending an RF pulse over a non-constant gradient (e.g., for realizing a VERSE pulse) may be implemented as an IRecursiveEventHandler. As an example, a time slice receiving data over a constant gradient may be implemented as an IRecursiveEventHandler. As another example, a time slice receiving data over a not constant gradient (e.g., ramp sampling on trapezoidal or sinusoidal ramps) may be implemented as an IRecursiveEventHandler. As yet another example, time slice switching 0^{th} order or higher order gradient moments between two gradient vectors may be implemented as an IRecursiveEventHandler. As an example, time slice receiving data on a spiral or any other non-Cartesian 2d or 3d readout may be implemented as an IRecursiveEventHandler. As another example, a time slice sending a two-dimensional (2D) or three-dimensional (3D) excitation pulse over a complex gradient waveform may be implemented as an IRecursiveEventHandler. As yet another example, a command for loading a new library containing one or more IRecursiveEventHandler implementations at run time may be implemented as an IRecursiveEventHandler. The event type would be a library name. As an example, a command for mapping a UUID to a new or existing IRecursiveEventHandler implementation may be implemented as an IRecursiveEventHandler.

Generally, there are different groups of IRecursiveEventHandler implementations. In a first group, the method may be run on a particular MR scanner or on computer of the MR scanner. Alternatively, the method may be run on any computing entity, associated with the MR scanner on behalf of the scanner, so that the scanner will be provided with the result data to execute the sequence. In the first group, the IRecursiveEventHandler is implemented specifically for the MR scanner system architecture the IRecursiveEventHandler is supposed to run on. In a second group, the method may be decoupled from a particular MR scanner. The method may be run on any computing entity. In this second group, the method may, for example, be run on an on-premise computer for several scanners in a hospital or may even be run in the cloud for several hospitals. In the second group, the IRecursiveEventHandler will just take an input event and create new events and pass the new events recursively to IRecursiveEventHandler. These implementations will be completely independent of the underlying system architecture and are not hardware- and scanner-specific, and may be regarded as stable software components.

The standardized serialization format may be, but does not necessarily have to be, binary. The standardized serialization format may also be some other standardized format like JSon or XML. The unique identifier is to be visible separately from the content. The transport layer is to be able to transport the data. It is solely the receiver (e.g., a specific IRecursiveEventHandler implementation) that is to be able to de-serialize the content.

An event handler only shows direct dependency to the specific control data types the event handler processes and to the control data types the event handler passes to the recursive event handler and not to other data (e.g., data deeper in a calling tree structure).

In the following, the present embodiments will be further described with respect to the figures.

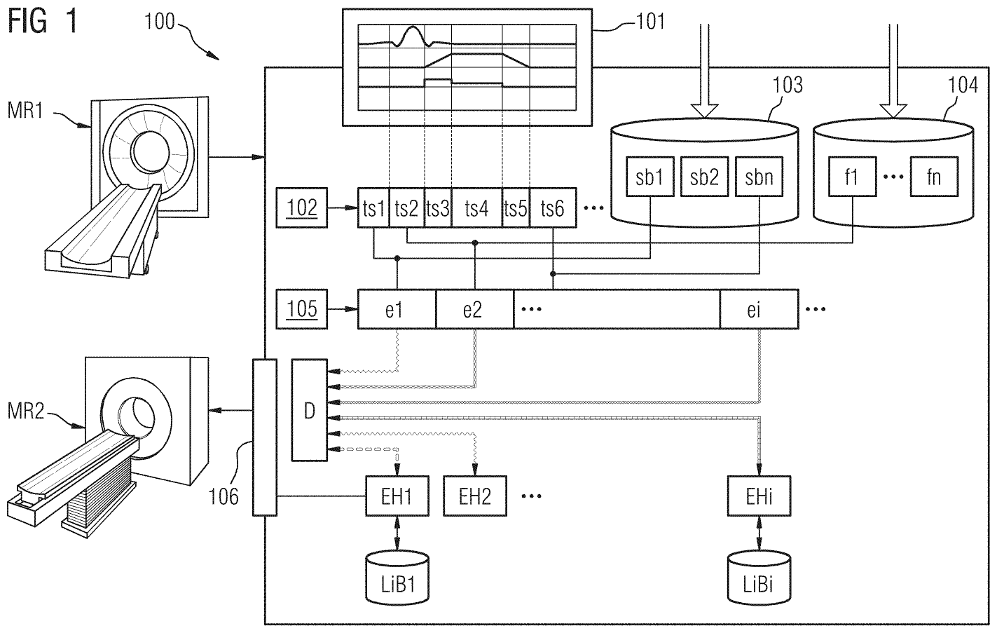

FIG. 1 schematically and exemplarily shows a system 100 in an overview manner. A first magnetic resonance (MR) scanner MR1 (e.g., a first scanner) and a second MR scanner MR2 (e.g., a second scanner) are provided. The first scanner MR1 and the second scanner MR2 may be of different type (e.g., different software, hardware, and/or different physical capabilities). The scanners MR are controlled by a computing unit, which is represented in FIG. 1 with the biggest rectangle on the right side, which is together with the associated MR scanners MR a major part of the system 100 and has respective digital interfaces to the scanners and to other data entries (e.g., for receiving a sequence or a stream of events).

For example, the computing unit, described above, includes an input interface 101 that is configured for receiving an MR sequence. Additionally, optional further interfaces may be provided (e.g., for receiving and storing a set of sequence block control data types sb in a storage 103 and/or for receiving and storing a set of fundamental control data types that are used to build fundamental control data types fin another storage 104).

A partitioning unit 102 is provided for partitioning the received MR sequence into time slices ts (in FIG. 1, ts1, ts2, . . . ts6 . . . ).

A mapping unit 105 that is configured for mapping each time slice ts to an instance of a sequence block control data type sb or to an instance of a fundamental control data type, which is of fundamental control data type f for providing a stream of events e representing the received MR sequence, is provided. In other words, the received MR sequence via interface 101 and the times slices, provided by the partitioning unit 102, serves a source or input for the stream of events, which is provided by the mapping unit 105.

An event handler EH is provided for each event e, where each of the event handlers EH1, EH2, . . . EHi is adapted for processing one or more specific events e1, e2, . . . ei. An event handler EH may be provided with a storage and an interface for receiving and storing of new event handlers.

A dispatcher D is provided and adapted for reading each event e in the provided stream of events and passing each event e together with the dispatcher D itself to an appropriate event handler EH. This may be done by processing the UUID markings of the events in correspondence to the respective event handlers EH. The dispatcher D may be called recursively by the event handlers EH (e.g., in FIG. 1, may be represented with the double headed arrows form dispatcher D to event handler EH) for generating control commands to be used for execution on the MR scanner. The control commands may be provided on an output interface 106. The control commands may be used to control a second MR scanner MR2, which may have different properties than the first scanner MR1.

The event handlers EH may be dedicated to a specific task. Some event handlers are configured to process fundamental control data types f, and others may be configured to process other data (e.g., sequence block control data types sb). Only the event handlers EH for the fundamental control data types f are directly communicating with the output interface 106. The dispatcher D usually does not address the MR scanner MR, because the dispatcher D has no knowledge about the scanner. The distinction between the different event handler types (e.g., one for fundamental control data types f and another one for sequence block control data types sb) is not explicitly represented in the figures for the sake of clarity.

In alternative embodiments, the storages 103, 104 may be provided as a separate instance out of the system 100 and to connect them via network connection or interfaces. Further, it is possible to integrate the partitioning unit 102 and the mapping unit 105 into one common unit to interact with the dispatcher D.

As shown in FIG. 1, the event handlers EH may call libraries LIB to process the event e.

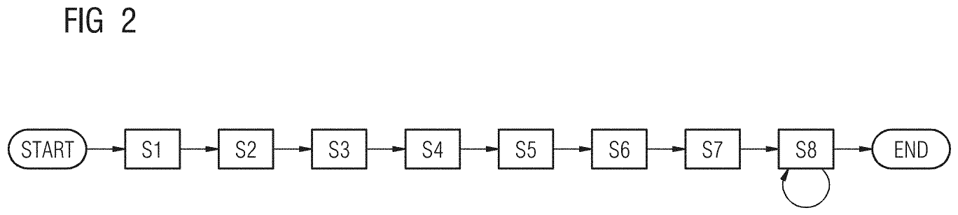

FIG. 2 shows a flow chart of one embodiment of a method. After START of a procedure, in act 51, an MR sequence is received. This may be executed via the input interface 101 or by reading from a local or external storage. In act S2, the received or read-in MR sequence is serialized and partitioned into time slices ts1, ts2, . . . tsn.

In act S3, an extendable set of sequence block control data types are defined. A sequence block control data type represents a formalized and a self-contained description of a time slice ts of the MR sequence. In act S4, a set of fundamental control data types f (e.g., corresponding to low-level commands) is provided. The fundamental control data types f may be stored in a local storage 104. In act S5, a mapping is performed. Each time slice ts is mapped to an instance of a sequence block control data type sb for providing a stream of events e1, e2, e3, . . . ei, representing the received MR sequence. In act S7, a set of event handlers EH is provided, where each of the event handlers EH is adapted for processing at least one event e. In act S8, the dispatcher is accessed for reading each event e in the provided stream of events and passing each event e together with the dispatcher D itself to an appropriate event handler EH, so that the dispatcher D may be called recursively by the event handlers EH for generating control commands to be used for execution on the MR scanners MR1, MR2.

In a more particular embodiment, the units 101, 102, 103, 104 and 105 of the system may be outsourced, and an input interface for receiving a stream of events e1, . . . ei, which may, for example, be provided by an external mapping unit 105, the dispatcher D, and the event handlers EH, is only included in the system 100. Since the event handlers EH may be extended easily by adding new event handlers, the system is highly scalable.

The concept of one or more of the present embodiments shows several advantages. The software architecture involved is minimal. The IRecursiveEventHandler is the only non-data software interface. The standardized control data format is not an interface and has no functionality. The standardized control data format only represents pure data collections that may easily be defined and understood. New IRecursiveEventHandler implementations and new control data types may be added to the system at any time and loaded during runtime without changing prior implementations (e.g., also by research customers or 3rd Party companies). Once a control data type is released, the behavior of the MR scanner MR may be declared as stable for all future when the MR scanner receives the released type. The implementation may change, but the scanner behavior remains. For example, this provides that all implementations of IRecursiveEventHandler that only use the recursive feature (e.g., do not implement anything and just create new events and do not process fundamental control data types) may be regarded as completely independent of a specific MR scanner architecture. The given concept defines the highest possible level of abstraction between the description of a pulse sequence and an actual execution on a hardware and software platform.

The interfaces of the system may be embodied as a hardware interface or as a software interface (e.g., PCI-Bus, USB or Firewire). In general, the system may include hardware elements and software elements (e.g., a microprocessor, a field programmable gate array (FPGA), or an application specific integrated circuit (ASIC)). The storages 103, 104 may be memory units and may be embodied as non-permanent main memory (e.g., random access memory (RAM)) or as permanent mass storage (e.g., hard disk, USB stick, SD card, sol-id state disk).

The system 100 and the MR scanners MR may be connected via a network that may be realized as a local area network (LAN) (e.g., a WiFi network) or any other local connection (e.g., via Bluetooth or universal serial bus (USB). The network may also be realized as a wide area network (WAN). For example, the network may be identical with the Internet. The network may alternatively also be realized as a virtual private network (VPN).

Other variations to the disclosed embodiments may be understood and effected by those skilled in the art in practicing the claimed invention from a study of the drawings, the disclosure, and the appended claims. In the claims, the word “comprising” does not exclude other elements or steps, and the indefinite article “a” or “an” does not exclude a plurality.

A single unit or device may fulfil the functions of several items recited in the claims. The mere fact that certain measures are recited in mutually different dependent claims does not indicate that a combination of these measures cannot be used to advantage.

The system for generating at least part of a pulse sequence to be executed on a magnetic resonance scanner in accordance with the method as described above may be implemented as program code of a computer program and/or as dedicated hardware.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

Wherever not already described explicitly, individual embodiments, or corresponding individual aspects and features, described in relation to the drawings may be combined or exchanged with one another without limiting or widening the scope of the described invention, whenever such a combination or exchange is meaningful and in the sense of this invention. Advantages that are described with respect to a particular embodiment of the present invention or with respect to a particular figure are, wherever applicable, also advantages of other embodiments of the present invention.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A computer implemented method for generating control commands for execution on a magnetic resonance (MR) scanner, the method comprising:
receiving a provided stream of events representing an MR sequence;
providing an event handler for each event of the provided stream of events, wherein each of the event handlers (EH) is adapted for processing at least one event of the provided stream of events;
accessing a dispatcher for reading each event of the provided stream of events and passing each event of the provided stream of events together with the dispatcher to an appropriate event handler of the provided event handlers, so that the dispatcher is recursively callable by the event handlers for generating control commands to be used for execution on the MR scanner.

2. The method of claim 1, further comprising providing the stream of events, providing the stream of events comprising:
receiving the MR sequence via an input interface;
partitioning the MR sequence into a sequence of time slices, each time slice of the sequence of time slices being a continuous uninterrupted period of time within the MR sequence;
defining an extendable set of sequence block control data types, wherein a sequence block control data type of the defined extendable set of sequence block control data types represents a formalized and a self-contained description of a time slice of the sequence of time slices of the MR sequence; and
mapping each time slice of the sequence of time slices to one instance of a sequence block control data type of the defined extendable set of sequence block control data types or to at least one instance of a fundamental control data type.

3. The method of claim 1, wherein each of the event handlers is operable to create new events.

4. The method of claim 1, wherein the respective event in the stream of events is a package consisting of a uniform unique identifier (UUID), a data size in bytes, and event data, and
wherein the UUID and the data size are accessible separately from the event data.

5. The method of claim 1, wherein providing an event handler for each event of the provided stream of events comprises including a uniform and unique identifier in the respective event and within the dispatcher, assigning the unique identifier to the respective event handler.

6. The method of claim 1, wherein recursive calls to the dispatcher are usable for applying a composite design pattern or a tree structure for generating a complete MR sequence.

7. The method of claim 1, wherein the generated control commands are scanner-independent, and
wherein only fundamental control data types have to be translated into scanner-specific control commands by the respective event handler.

8. The method of claim 1, wherein the dispatcher and the respective event handler are both implementing a same interface for sequence block execution.

9. The method of claim 1, wherein a definition of a set of sequence block control data types is based on a provided abstract model for MR sequences, and
wherein the model is baseable on an extendable set of fundamental control data types.

10. The method of claim 1, wherein a set of fundamental control data types and a set of sequence block control data types are extendable by adding new control data types without affecting existing code, the new control data types being fundamental control data types or sequence block control data types.

11. The method of claim 1, wherein a set of the event handlers is extendable by adding new event handlers without affecting existing code.

12. The method of claim 2, wherein the mapping, the accessing of the dispatcher, and recursively calling the dispatcher are implemented by an IRecursiveEventHandler interface.

13. The method of claim 1, wherein the stream of events is providable by an exchangeable software Plug-In into an MR control software executing the dispatcher.

14. A computing device for generating control commands for execution on a magnetic resonance (MR) scanner, the computing device comprising:
an input interface configured to receive a provided stream of events representing a received MR sequence;
an event handler for each event of the provided stream of events, wherein each of the event handlers is configured to process one or more specific events; and
a dispatcher configured to:
read each event of the provided stream of events; and
pass each event of the provided stream of events together with the dispatcher to an appropriate event handler, so that the dispatcher is callable recursively by the event handlers for generating control commands to be used for execution on the MR scanner.

15. The computing device of claim 14, further comprising:
an input interface configured to receive an MR sequence;
a partitioner configured to partition the received MR sequence into a sequence of time slices, each time slice of the sequence of time slices being a continuous uninterrupted period of time within the MR sequence;
a storage device configured to provide an extendable set of sequence block control data types, wherein a sequence block control data type of the extendable set of sequence block control data types represents a formalized and self-contained description of a time slice of the sequence of time slices of the MR sequence; and
a mapper configured to map each time slice of the sequence of time slices to at least one instance of a sequence block control data type of the extendable set of sequence block control data types or to at least one instance of a fundamental control data type for providing the stream of events representing the received MR sequence.

16. A system for generating control commands for execution on a magnetic resonance (MR) scanner, the system comprising:
at least one MR scanner to be operated with control commands to be generated; and
a computing device for generating the control commands for execution on the at least one MR scanner, the computing device comprising:
an input interface configured to receive a provided stream of events representing a received MR sequence;
an event handler for each event of the provided stream of events, wherein each of the event handlers is configured to process one or more specific events; and
a dispatcher configured to:
read each event of the provided stream of events; and
pass each event of the provided stream of events together with the dispatcher to an appropriate event handler, so that the dispatcher is callable recursively by the event handlers for generating control commands to be used for execution on the MR scanner.

17. In a non-transitory computer-readable storage medium that stores instructions executable by one or more processors to generate control commands for execution on a magnetic resonance (MR) scanner, the instructions comprising:
receiving a provided stream of events representing an MR sequence;
providing an event handler for each event of the provided stream of events, wherein each of the event handlers (EH) is adapted for processing at least one event of the provided stream of events;
accessing a dispatcher for reading each event of the provided stream of events and passing each event of the provided stream of events together with the dispatcher to an appropriate event handler of the provided event handlers, so that the dispatcher is recursively callable by the event handlers for generating control commands to be used for execution on the MR scanner.
